(19) United States
(12) Patent Application Publication
(10) Pub. No.: US 2018/0008665 A1
(43) Pub. Date: Jan. 11, 2018

(54) METHODS OF TREATING AND PREVENTING GOUT AND LEAD NEPHROPATHY

(71) Applicant: CanTrust LifeScience Corp., Vancouver (CA)

(72) Inventors: Grace Qiao, Vancouver (CA); Lili Zhang, Vancouver (CA); Feng Qiao, Vancouver (CA); Longjun Dai, Vancouver (CA)

(73) Assignee: CanTrust LifeScience Corp., Vancouver (CA)

(21) Appl. No.: 15/581,439

(22) Filed: Apr. 28, 2017

Related U.S. Application Data
(60) Provisional application No. 62/358,306, filed on Jul. 5, 2016.

(51) Int. Cl. A61K 38/06; A61K 9/00

(52) U.S. Cl. CPC A61K 38/063; A61K 9/0053

(57) ABSTRACT

Disclosed here is a novel use of a pharmaceutical composition comprising glutathione or its pharmaceutically acceptable salts or derivatives as active ingredient, in the forms of tablets, capsules, powder, spray and/or liquid, through oral administration, intervene injection, inhale and/or skin transmission, to treat and prevent gout and lead nephropathy. Embodiments of gout and lead nephropathy treatable by the pharmaceutical composition described herein include, without limitation, gout, kidney stones, tophi, hyperuricemia, blood lead, renal disease (lead nephropathy), complications thereof, as well as other conditions caused by lifestyle, genetics, medical conditions, and blood lead level. Symptoms treatable may include inflammatory arthritis, which typically involves a red, tender, hot, and swollen joint.

### CROSS REFERENCE TO RELATED APPLICATIONS

This application claims priority to U.S. Provisional Application No. 62/358,306, filed Jul. 5, 2016, which is hereby incorporated by reference in its entirety.

### BACKGROUND

Gout is a metabolic syndrome resulting from deposition of uric acid crystals in tissues and fluids within the body. This process is caused by an overproduction and/or under excretion of uric acid. Certain common medications, lead, alcohol, and dietary foods are known to be contributory factors. Acute gout typically manifests itself as an acutely red, hot, and swollen joint with excruciating pain. These acute gouty flare-ups respond well to treatment with oral anti-inflammatory medicines and may be prevented with medication and diet changes. Recurrent bouts of acute gout can lead to a degenerative form of chronic arthritis called gouty arthritis.

Gout causes a common form of inflammatory arthritis, which is the most common inflammatory arthritis among men. Gout may remit for long periods, followed by flares for days to weeks, or can become chronic.

According to the Center for Disease Control (CDC), the prevalence of gout among U.S. adults in 2007-2008 was 3.9% (8.3 million individuals). The prevalence of gout among men was 5.9% (6.1 million), and the prevalence among women was 2.0% (2.2 million). The prevalence of gout increased over the past 2 decades by an estimate of 1.2 percentage points. The CDC estimated that medical expenditures attributable to gout in 2007-2008 were $2,805 per person or $7.7 billion overall (24% of all-cause gout expenditures).

Existing medications for treating gout primarily attempt to relieve symptoms including inflammation. Xanthine oxidase inhibitors have been considered as the first line drugs for the treatment of gout. Allopurinol (Zyloprim, a xanthine oxidase inhibitor), a medication for gout on the WHO Model List of Essential Medicines, does not alleviate acute attacks of gout. In addition, there is controversy over whether allopurinol actually makes acute gout attacks worse initially. Allopurinol also has two disadvantages. First, its dosing is complex. Second, some patients are hypersensitive to the drug and require careful monitoring. Allopurinol has rare but potentially fatal adverse effects involving the skin. One study tested on 57 men with crystal-proven gout concluded that allopurinol initiation during an acute gout attack caused no significant difference in daily pain, recurrent flares, or inflammatory markers.

Other treatment options for acute gout include nonsteroidal anti-inflammatory drugs (e.g., NSAIDs), colchicine and steroids, with the initial aim of treatment being alleviating the symptoms of an acute attack. Colchicine, a second-line treatment, is an alternative for those who are unable to tolerate NSAIDs in gout. Long-term (prophylactic) regimens of oral colchicine are contraindicated in patients with advanced renal failure (including those on dialysis). About 10-20 percent of a colchicine dose is excreted unchanged by the kidney, and it is not removable by hemodialysis. Cumulative toxicity is a high probability in this clinical setting, and a severe neuromyopathy may result.

To date, there is no curative treatment for gout, and no therapy has been demonstrated to significantly delay the progression of gout and lead nephropathy.

### SUMMARY

Disclosed here is a novel use of a pharmaceutical composition comprising glutathione as active ingredient for treating and preventing gout and lead nephropathy.

Accordingly, one aspect of the invention relates to a method for treating or preventing gout and lead nephropathy, comprising administrating to a subject in need thereof a pharmaceutical composition comprising a therapeutically effective amount of glutathione or a pharmaceutically acceptable salt thereof.

Another aspect of the invention relates to a method for treating a disease or condition associated with an elevated uric acid or urate level (e.g., hyperuricemia), comprising administrating to a subject in need thereof a pharmaceutical composition comprising a therapeutically effective amount of glutathione or a pharmaceutically acceptable salt thereof.

A further aspect of the invention relates to a method for treating a disease or condition selected from the group consisting of chronic gouty arthritis, acute inflammatory arthritis, uric acid nephropathy, kidney stones, tophi, and hyperuricemia, comprising administrating to a subject in need thereof a pharmaceutical composition comprising a therapeutically effective amount of glutathione or a pharmaceutically acceptable salt thereof.

An additional aspect of the invention relates to a method for reducing serum uric acid or urate level in vivo, comprising administrating to a subject in need thereof a pharmaceutical composition comprising a therapeutically effective amount of glutathione or a pharmaceutically acceptable salt thereof.

Yet another aspect of the invention relates to a method for increasing uric acid excretion and/or reducing uric acid production in vivo, comprising administrating to a subject in need thereof a pharmaceutical composition comprising a therapeutically effective amount of glutathione or a pharmaceutically acceptable salt thereof.

Yet a further aspect of the invention relates to a method for reducing blood lead level in vivo, comprising administrating to a subject in need thereof a pharmaceutical composition comprising a therapeutically effective amount of glutathione or a pharmaceutically acceptable salt thereof.

Yet a further aspect of the invention relates to a method for reducing serum creatinine level in vivo, comprising administrating to a subject in need thereof a pharmaceutical composition comprising a therapeutically effective amount of glutathione or a pharmaceutically acceptable salt thereof.

Yet an additional aspect of the invention relates to a pharmaceutical composition comprising a therapeutically effective amount of glutathione or a pharmaceutically acceptable salt thereof as active ingredient.

In some embodiments, the subject is a human. In some embodiments, the subject is a non-human animal.

In some embodiments, the method comprises administrating to the subject about 1-20 grams, or about 2-15 grams, or about 3-10 grams, or about 2-5 grams, or about 2-3 grams, of glutathione per day.

In some embodiments, the pharmaceutical composition comprises at least about 3 wt. %, or at least about 5 wt. %, or at least about 10 wt. %, or at least about 15 wt. %, or at least about 20 wt. %, or at least about 30 wt. %, or at least about 40 wt. %, of glutathione.

In some embodiments, the pharmaceutical composition further comprises at least one pharmaceutically acceptable carrier or excipient.

In some embodiments, the pharmaceutical composition's active ingredient consists essentially of or consists of glutathione. In some embodiments, glutathione is the sole active ingredient of the pharmaceutical composition.

In some embodiments, the pharmaceutical composition is substantially or totally free of another active ingredient for treating gout and/or lead nephropathy. In some embodiments, the pharmaceutical composition is substantially or totally free of sophoridine.

In some embodiments, the method comprises administration of another medication for treating gout and/or lead nephropathy, such as an anti-gout agent (e.g., allopurinol, benzbromarone, colchicine, probenecid, and sulfinpyrazone), an anti-inflammatory agent (e.g., steroidal and nonsteroidal anti-inflammatory drugs including ibuprofen and prednisone), and/or an antioxidant.

In some embodiments, the method is substantially or totally free of administration of another medication for treating gout and/or lead nephropathy.

In some embodiments, the pharmaceutical composition is in the form of tablets, pills, capsules, powders, granules, suppositories, dragees, ointments, sprays, inhalants, emulsions, solutions, suspensions, syrups, and elixirs.

In some embodiments, the pharmaceutical composition is administrated by a route selected from the group consisting of oral, intravenous, intramuscular, intra-arterial, intramedullary, intrathecal, intraventricular, transdermal, subcutaneous, intraperitoneal, intranasal, enteral, topical, sublingual, and rectal administrations.

In some embodiments, the pharmaceutical composition is orally administrated about one to three times daily, or about one time daily, or about two times daily, or about three times daily.

In some embodiments, the serum uric acid or urate level in the subject is reduced by at least about 5%, or at least about 10%, or at least about 20%, or at least about 30%, or at least about 40%, or at least about 50%, after 28 days of treatment. In some embodiments, the serum uric acid or urate level in the subject is reduced by at least about 5%, or at least about 10%, or at least about 20%, or at least about 30%, or at least about 40%, or at least about 50%, after 56 days of treatment. In some embodiments, the serum uric acid or urate level in the subject is reduced by at least about 5%, or at least about 10%, or at least about 20%, or at least about 30%, or at least about 40%, or at least about 50%, after 84 days of treatment.

In some embodiments, the blood lead level in the subject is reduced by at least about 5%, or at least about 10%, or at least about 20%, or at least about 30%, or at least about 40%, or at least about 50%, after 28 days of treatment. In some embodiments, the blood lead level in the subject is reduced by at least about 5%, or at least about 10%, or at least about 20%, or at least about 30%, or at least about 40%, or at least about 50%, after 56 days of treatment. In some embodiments, the blood lead level in the subject is reduced by at least about 5%, or at least about 10%, or at least about 20%, or at least about 30%, or at least about 40%, or at least about 50%, after 84 days of treatment.

In some embodiments, the serum creatinine level in the subject is reduced by at least about 5%, or at least about 10%, or at least about 20%, or at least about 30%, or at least about 40%, or at least about 50%, after 28 days of treatment. In some embodiments, the serum creatinine level in the subject is reduced by at least about 5%, or at least about 10%, or at least about 20%, or at least about 30%, or at least about 40%, or at least about 50%, after 56 days of treatment. In some embodiments, the serum creatinine level in the subject is reduced by at least about 5%, or at least about 10%, or at least about 20%, or at least about 30%, or at least about 40%, or at least about 50%, after 84 days of treatment.

In some embodiments, the volume of kidney stones or tophi in the subject is reduced by at least about 5%, or at least about 10%, or at least about 20%, or at least about 30%, or at least about 40%, or at least about 50%, after 28 days of treatment. In some embodiments, the volume of kidney stones or tophi in the subject is reduced by at least about 5%, or at least about 10%, or at least about 20%, or at least about 30%, or at least about 40%, or at least about 50%, after 56 days of treatment. In some embodiments, the volume of kidney stones or tophi in the subject is reduced by at least about 5%, or at least about 10%, or at least about 20%, or at least about 40%, or at least about 50%, or at least about 30%, or at least about 40%, or at least about 50%, after 84 days of treatment.

In some embodiments, the volume of uric acid or urate deposition in symptomatic joint or bursa of the subject is reduced by at least about 5%, or at least about 10%, or at least about 20%, or at least about 30%, or at least about 40%, or at least about 50%, after 28 days of treatment. In some embodiments, the volume of uric acid or urate deposition in symptomatic joint or bursa of the subject is reduced by at least about 5%, or at least about 10%, or at least about 20%, or at least about 30%, or at least about 40%, or at least about 50%, after 56 days of treatment. In some embodiments, the volume of uric acid or urate deposition in symptomatic joint or bursa of the subject is reduced by at least about 5%, or at least about 10%, or at least about 20%, or at least about 30%, or at least about 40%, or at least about 50%, after 84 days of treatment.

These and other features, together with the organization and manner of operation thereof, will become apparent from the following detailed description when taken in conjunction with the accompanying drawings.

### BRIEF DESCRIPTION OF THE DRAWINGS

FIG. 1 shows glutathione's effects on serum urate, over the course of 12 weeks (in intervals of 4 weeks). Graph data show reduction of serum urate level in subjects orally administered with glutathione tablet 3 times per day, 2 tablets at a time. Each tablet contains 450 mg of pharmaceutical grade of GSH powder.

FIG. 2 shows glutathione's effects on serum creatinine, over the course of 12 weeks (in intervals of 4 weeks). Graph data show reduction of serum creatinine level in subjects orally administered with glutathione tablet 3 times per day, 2 tablets at a time. Each tablet contains 450 mg of pharmaceutical grade of GSH powder.

FIG. 3 shows glutathione's effects on blood lead level, over the course of 12 weeks (in intervals of 4 weeks). Graph data show reduction of blood lead level in subjects orally administered with glutathione tablet 3 times per day, 2 tablets at a time. Each tablet contains 450 mg of pharmaceutical grade of GSH powder.

### DETAILED DESCRIPTION

Reference will now be made in detail to some specific embodiments of the invention contemplated by the inventors for carrying out the invention. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise.

### Pharmaceutical Formulations

Provided herein are a pharmaceutical composition comprising glutathione or its pharmaceutically acceptable salts or derivatives as active ingredient and use thereof in treating gout and/or lead nephropathy. Glutathione are administered in a therapeutically effective amount to patients suffering from gout and/or lead nephropathy. Glutathione provides a prolonged therapeutic benefit with negligible side effects. In contrast to existing anti-gout medications which are limited to management of symptoms, the glutathione-based medication described herein provides a curative treatment for patients suffering from gout and/or lead nephropathy.

Glutathione or its pharmaceutically acceptable salts or derivatives can be provided as active ingredient in a pharmaceutical composition. Depending on the intended mode of administration, the pharmaceutical composition can be in the form of solid, semi-solid or liquid dosage forms such as, for example, tablets, suppositories, pills, capsules, powders, liquids, and suspensions, preferably in a unit dosage form suitable for single administration of a precise dosage. The pharmaceutical composition can include a therapeutically effective amount of glutathione or its pharmaceutically acceptable salts or derivatives, in combination with at least one pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a pharmaceutical composition will depend upon the intended route of administration for the pharmaceutical composition.

The pharmaceutical composition described herein for parenteral injection may comprise one or more physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils and injectable organic esters such as ethyl oleate. Proper fluidity can be maintained, for example, by the use of a coating such as lecithin, by the maintenance of the required particle size in the case of dispersions and by the use of surfactants.

The pharmaceutical composition described herein may also comprise one or more adjuvants such as preserving, wetting, emulsifying, and dispensing agents. Prevention of the action of microorganisms can be achieved by various antibacterial and antifungal agents such as, for example, parabens, chlorobutanol, phenol, sorbic acid, and the like. Isotonic agents such as, for example, sugars, sodium chloride, and the like may also be included. Prolonged absorption of the injectable pharmaceutical form can be achieved by the use of absorption delaying agents such as, for example, aluminum monostearate and gelatin.

Solid dosage forms of the pharmaceutical composition described herein for oral administration include, for example, capsules, tablets, pills, powders, and granules. In such solid dosage forms, glutathione or its pharmaceutically acceptable salts or derivatives can be admixed with at least one inert customary excipient (or carrier), such as sodium citrate or dicalcium phosphate, or (1) fillers or extenders such as, for example, starches, lactose, sucrose, glucose, mannitol, and silicic acid, (2) binders such as, for example, carboxymethylcellulose, alignates, gelatin, polyvinylpyrrolidone, sucrose, and acacia, (3) humectants such as, for example, glycerol, (4) disintegrating agents such as, for example, agar-agar, calcium carbonate, potato or tapioca starch, alginic acid, certain complex silicates, and sodium carbonate, (5) solution retarders such as, for example, paraffin, (6) absorption accelerators such as, for example, quaternary ammonium compounds, (7) wetting agents such as, for example, cetyl alcohol, and glycerol monostearate, (8) adsorbents such as, for example, kaolin and bentonite, and/or (9) lubricants such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using excipients such as lactose, milk sugar, high molecular weight polyethyleneglycols, and the like.

Solid dosage forms such as tablets, dragees, capsules, pills, and granules can be prepared with coatings and shells, such as enteric coatings and other coatings known in the art. They may contain opacifying agents and can also be adapted to release the active compound(s) in a certain part of the intestinal tract in a delayed manner. Examples of embedding compositions that can be used include polymeric substances and waxes. The active compound(s) can also be in micro-encapsulated form, when appropriate, with one or more of the above-mentioned excipients.

Liquid dosage forms of the pharmaceutical composition described herein for oral administration include, for example, pharmaceutically acceptable emulsions, solutions, suspensions, syrups, and elixirs. In addition to the active compound(s), the liquid dosage forms may comprise one or more inert diluents commonly used in the art, such as water or other solvents, solubilizing agents, and emulsifiers such as, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents such as, for example, wetting, emulsifying, suspending, sweetening, flavoring, and/or perfuming agents.

Suspensions, in addition to the active compounds, may contain additional agents such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

The pharmaceutical compositions described herein for rectal administrations are optionally suppositories, which can be prepared by mixing the compounds with suitable non-irritating excipients or carriers such as, for example, cocoa butter, polyethyleneglycol or a suppository wax, which are solid at ordinary temperatures but liquid at body temperature and, therefore, would melt in the rectum or vaginal cavity to release the active component.

Dosage forms of the pharmaceutical composition described herein for topical administration include, for example, ointments, powders, sprays, and inhalants. Glutathione or its pharmaceutically acceptable salts or derivatives can be admixed under sterile conditions with a physiologically acceptable carrier and any preservatives, buffers, or propellants as may be required. Ophthalmic formulations, ointments, powders, and solutions are also contemplated as being within the scope of the pharmaceutical compositions described herein.

### Methods of Treating or Preventing Gout and Lead Nephropathy

Provided herein are methods of treating or preventing gout and lead nephropathy, comprising administering to a subject in need thereof an effective amount of glutathione or its pharmaceutically acceptable salts or derivatives. The term “effective amount,” when used to describe an amount of glutathione or its pharmaceutically acceptable salts or derivatives in the method described herein, refers to the amount of glutathione or its pharmaceutically acceptable salts or derivatives that achieves the desired pharmacological effect or other effects such as, for example, an amount that results in uric acid excretion increase and/or uric acid production reduction and/or blood lead reduction. The pharmaceutical compositions described herein are useful for treating gout or lead nephropathy in humans, including, without limitation, pediatric and geriatric populations, and in animals (e.g., veterinary applications). Optionally, the methods can be used to treat conditions associated with elevated uric acid levels, including chronic gouty arthritis, acute inflammatory arthritis, uric acid nephropathy, kidney stones, or tophi.

Further described herein are methods of increasing uric acid excretion and/or reducing uric acid production and reducing blood lead level, comprising administering to a subject in need thereof an effective amount of glutathione or its pharmaceutically acceptable salts or derivatives. Optionally, the methods can further comprise selecting a subject having gout or lead nephropathy.

The methods described herein can further comprise administering to the subject a second therapeutic agent. Thus, the provided compositions and methods can include one or more additional agents. The one or more additional agents, and the glutathione or its pharmaceutically acceptable salts or derivatives, can be administered in any order, including concomitant, simultaneous, or sequential administration. Sequential administration can be temporally spaced order of up to several days apart. The methods can also include more than a single administration of the one or more additional agents and/or the glutathione or its pharmaceutically acceptable salts or derivatives or prodrugs. The administration of the one or more additional agents, and the glutathione or its pharmaceutically acceptable salts or derivatives or prodrugs, can be through the same or different routes and concurrently or sequentially.

The additional therapeutic agents can include, but are not limited to, anti-gout agents. For example, the anti-gout agent can be allopurinol, benzbromarone, colchicine, probenecid, or sulfinpyrazone. Moreover, the additional therapeutic agents can include anti-inflammatory agents. Examples of suitable anti-inflammatory agents include, for example, steroidal and nonsteroidal anti-inflammatory drugs (e.g., ibuprofen and prednisone). Furthermore, the additional therapeutic agents can include antioxidants. Examples of suitable antioxidants include, for example, alpha-tocopherol, beta-carotene, butylated hydroxyanisole (BHA), butylated hydroxytoluene, caffeic acid, lutein, lycopene, selenium, tert-butylhydroquinone, Vitamin A, Vitamin C, and Vitamin E. Further examples of suitable antioxidants include putative antioxidant botanticals, such as, for example, grape seeds, green tea, Scutellaria baicalensis, American ginseng, ginkgo biloba, and the like.

Any of the aforementioned therapeutic agents can be used in any combination with the compositions described herein. Combinations are administered either concomitantly (e.g., as an admixture), separately but simultaneously, or sequentially. Thus, the term combination is used to refer to concomitant, simultaneous, or sequential administration of two or more agents.

The methods and pharmaceutical compositions described herein are useful for both prophylactic and therapeutic treatment. For prophylactic use, a therapeutically effective amount of glutathione or its pharmaceutically acceptable salts or derivatives are administered to a subject prior to onset (e.g., before obvious signs of gout or hyperuricemia), during early onset (e.g., upon initial signs and symptoms of gout or hyperuricemia), or after the development of gout or hyperuricemia. Prophylactic administration can occur for several days to years prior to the manifestation of symptoms of gout or hyperuricemia. Therapeutic treatment can comprise administering to a subject in need thereof a therapeutically effective amount of glutathione or its pharmaceutically acceptable salts or derivatives after gout or hyperuricemia is diagnosed.

The methods and pharmaceutical compositions described herein are also useful in increasing uric acid excretion activity in kidney system and and/or reducing uric acid production in blood system, optionally in vivo.

### Gout Classification Criteria and Parameters

The classification criteria and parameters of gout may include one or more of the following:

Characteristics of symptomatic episode(s) ever: (i) erythema overlying affected joint (patient-reported or physician-observed); (ii) can't bear touch or pressure to affected joint; (iii) great difficulty with walking or inability to use affected joint.

Time-course of episode(s) ever: Presence (ever) of ≧2,irrespective of anti-inflammatory treatment: (i) time to maximal pain <24 hours; (ii) resolution of symptoms in ≦14 days; (iii) complete resolution (to baseline level) between symptomatic episodes.

Additional criteria and parameters include: (A) serum urate (measured by uricase method and ideally should be scored at a time when the patient was not taking urate-lowering treatment and was beyond 4 weeks from the start of an episode (i.e., during intercritical period), and retest under those conditions if practicable, with the highest value irrespective of timing scored), (B) synovial fluid analysis of a symptomatic (ever) joint or bursa (should be assessed by a trained observer), (C) imaging evidence of urate deposition in symptomatic (ever) joint or bursa (e.g., ultrasound evidence of double-contour sign or DECT demonstrating urate deposition), (D) imaging evidence of gout-related joint damage (e.g., conventional radiography of the hands and/or feet demonstrating at least one erosion), (E) blood lead level, and (F) serum creatinine level.

### WORKING EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

Seven (7) subjects with gout and hyperuricemia were examined. Specifically, these seven subjects were part of an open-label, twelve (12) weeks study in which they received 2700 mg daily (450 mg/tablet, 2 tablets/time, 3 times/day) of (2S)-2-Amino-4-{[(1R)-1-[(carboxymethyl)carbamoyl]-2-sulfanylethyl]carbamoyl}butanoic acid (hereinafter “CT° 1”).

The subjects were predominantly male, with a mean age of 53.14±5.46 years. Five (5) of the subjects had gout for more than 10 years, two (2) subjects had hyperuricemia for between 1 to 5 years. Overall, the subjects were classified as renal underexcretion.

### Example 1

### CT°I Induced Reduction of Serum Urate

CT°I participates directly in the neutralization of free radicals and reactive oxygen compounds. The effects of CT°I on serum urate include at least two possible aspects: 1) directly reduce the production of urate through the interaction with xanthine oxidase; 2) indirectly reduce serum urate level through CT°I induced improvement of renal function.

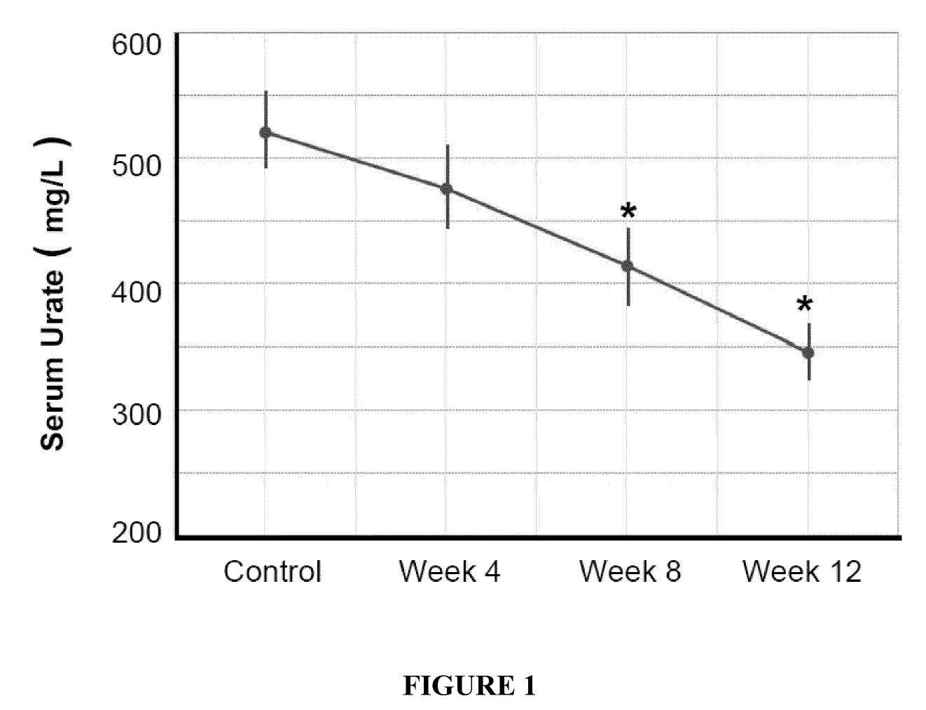

The mean serum urate of the tested subjects at their first visit was 521.07±40.11 mg/L, and the mean serum urate after 12 weeks of treatment was 346.46±21.17 mg/L, as shown in FIG. 1. This represents a reduction of serum urate of 33.51%. The reduction of serum urate appeared significantly different after 8 weeks of treatment (p<0.05).

### Example 2

### CT°I Induced Renal Function Improvement

All of the subjects were classified as renally impaired (glomerular filtration rate <80 ml/minute). A serum creatinine test measures the level of creatinine in the blood and provides an estimate of glomerular filtration rate that indicates the excretory function of the kidney. If the kidney functions improperly, an increased level of creatinine may accumulate in the blood.

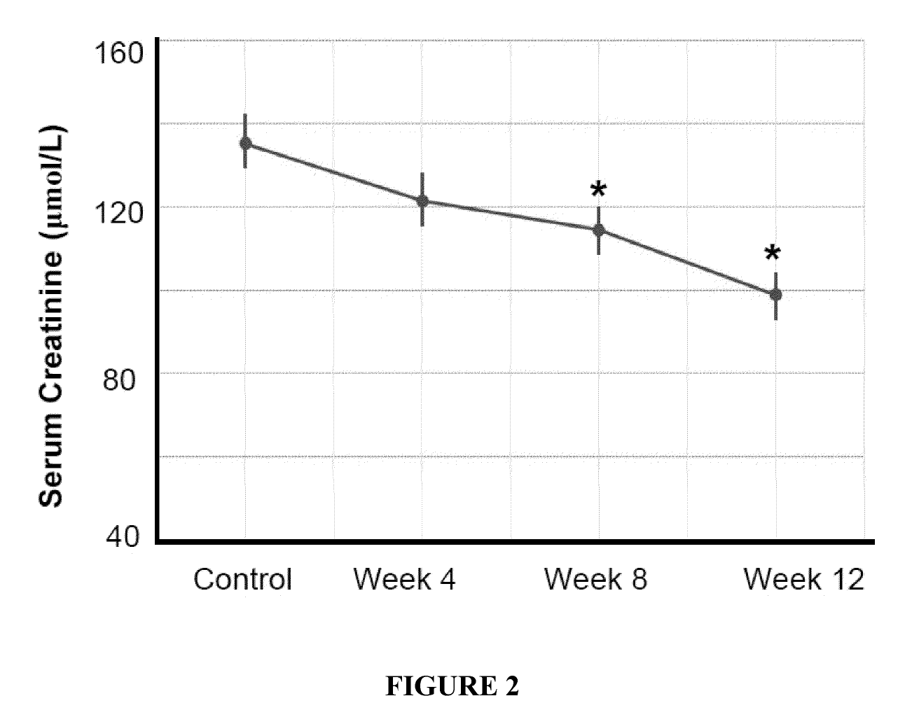

The mean serum creatinine of the tested subjects at their first visit was 135.29±6.89 μmol/L, and the mean serum creatinine after 12 weeks of treatment was 99.00±4.45 μmol/L, as shown in FIG. 2. This represents a reduction of serum creatinine of 26.82%. The reduction of serum creatinine appeared significantly different after 8 weeks of treatment (p<0.05).

### Example 3

### CT°I Induced Reduction of Blood Lead Level

Lead is toxic to the kidney. The accumulated heavy metal ions (including lead) harm the body primarily through their potent oxidative damage to major organs, especially kidney and liver. CT°I protects the body from heavy metal ion poisoning mainly through its direct chelation and its intensive antioxidant effects.

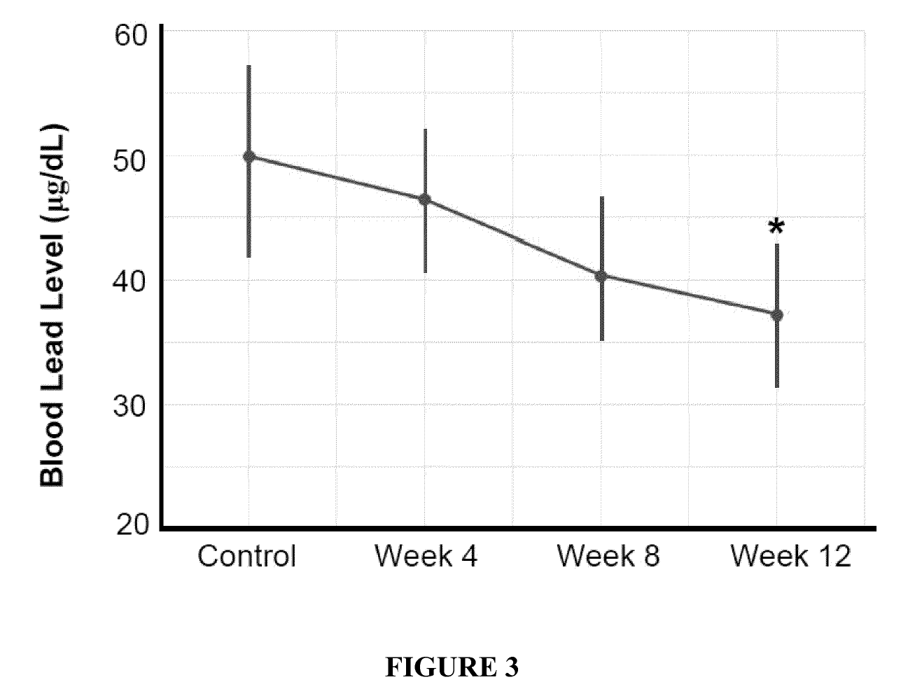

The mean blood lead level of the tested subjects at their first visit was 49.90±8.41 μg/dL, and the mean blood lead level after 12 weeks of treatment was 37.26±6.29 μg/dL, as shown in FIG. 3. This represents a reduction of blood lead level of 25.33%. The reduction of blood lead level appeared significantly different after 12 weeks of treatment (p<0.05).

### Additional Embodiments

Embodiment 1—A method for treating gout and/or lead nephropathy, comprising administrating to a subject in need thereof a pharmaceutical composition comprising a therapeutically effective amount of glutathione or a pharmaceutically acceptable salt thereof.

Embodiment 2—A method for treating a disease or condition associated with an elevated uric acid or urate level (e.g., hyperuricemia), comprising administrating to a subject in need thereof a pharmaceutical composition comprising a therapeutically effective amount of glutathione or a pharmaceutically acceptable salt thereof.

Embodiment 3—A method for treating a disease or condition selected from the group consisting of chronic gouty arthritis, acute inflammatory arthritis, uric acid nephropathy, kidney stones, tophi, and hyperuricemia, comprising administrating to a subject in need thereof a pharmaceutical composition comprising a therapeutically effective amount of glutathione or a pharmaceutically acceptable salt thereof.

Embodiment 4—A method for reducing serum uric acid or urate level in vivo, comprising administrating to a subject in need thereof a pharmaceutical composition comprising a therapeutically effective amount of glutathione or a pharmaceutically acceptable salt thereof.

Embodiment 5—A method for increasing uric acid excretion and/or reducing uric acid production in vivo, comprising administrating to a subject in need thereof a pharmaceutical composition comprising a therapeutically effective amount of glutathione or a pharmaceutically acceptable salt thereof.

Embodiment 6—A method for reducing blood lead level in vivo, comprising administrating to a subject in need thereof a pharmaceutical composition comprising a therapeutically effective amount of glutathione or a pharmaceutically acceptable salt thereof.

Embodiment 7—A method for reducing serum creatinine level in vivo, comprising administrating to a subject in need thereof a pharmaceutical composition comprising a therapeutically effective amount of glutathione or a pharmaceutically acceptable salt thereof.

Embodiment 8—The method of any of embodiments 1-7, wherein the subject is a human.

Embodiment 9—The method of any of embodiments 1-8, wherein the method comprises administrating to the subject about 1-20 grams of glutathione per day.

Embodiment 10—The method of any of embodiments 1-9, wherein the pharmaceutical composition comprises at least about 3 wt. % of glutathione.

Embodiment 11—The method of any of embodiments 1-10, wherein the pharmaceutical composition further comprises at least one pharmaceutically acceptable carrier or excipient.

Embodiment 12—The method of any of embodiments 1-11, wherein the pharmaceutical composition's active ingredient consists essentially of glutathione.

Embodiment 13—The method of any of embodiments 1-12, wherein the pharmaceutical composition is substantially free of another active ingredient for treating gout and/or lead nephropathy.

Embodiment 14—The method of any of embodiments 1-13, wherein the pharmaceutical composition is substantially free of sophoridine.

Embodiment 15—The method of any of embodiments 1-14 wherein the pharmaceutical composition is in the form of tablets, pills, capsules, powders, granules, suppositories, dragees, ointments, sprays, inhalants, emulsions, solutions, suspensions, syrups, and elixirs.

Embodiment 16—The method of any of embodiments 1-15, wherein the pharmaceutical composition is administrated by a route selected from the group consisting of oral, intravenous, intramuscular, intra-arterial, intramedullary, intrathecal, intraventricular, transdermal, subcutaneous, intraperitoneal, intranasal, enteral, topical, sublingual, and rectal administrations.

Embodiment 17—The method of any of embodiments 1-16, wherein the pharmaceutical composition is orally administrated one to three times daily.

Embodiment 18—The method of any of embodiments 1-17, wherein the serum uric acid or urate level in the subject is reduced by at least about 10% after 84 days of treatment.

Embodiment 19—The method of any of embodiments 1-18, wherein the blood lead level in the subject is reduced by at least about 10% after 84 days of treatment.

Embodiment 20—The method of any of embodiments 1-19, wherein the serum creatinine level in the subject is reduced by at least about 10% after 84 days of treatment.

Embodiment 21—The method of any of embodiments 1-20, wherein the volume of kidney stones or tophi in the subject is reduced by at least about 10% after 84 days of treatment.

Embodiment 22—The method of any of embodiments 1-21, wherein the volume of uric acid deposition in symptomatic joint or bursa of the subject is reduced by at least about 10% after 84 days of treatment.

Embodiment 23—A pharmaceutical composition comprising a therapeutically effective amount of glutathione or a pharmaceutically acceptable salt thereof.

Embodiment 24—The pharmaceutical composition of embodiments 23, wherein the pharmaceutical composition comprises about 0.1-10 grams of glutathione.

Embodiment 25—The pharmaceutical composition of any of embodiments 23-24, wherein the pharmaceutical composition comprises at least about 3 wt. % of glutathione.

Embodiment 26—The pharmaceutical composition of any of embodiments 23-25, wherein the pharmaceutical composition further comprises at least one pharmaceutically acceptable carrier or excipient.

Embodiment 27—The pharmaceutical composition of any of embodiments 23-26, wherein the pharmaceutical composition's active ingredient consists essentially of glutathione.

Embodiment 28—The pharmaceutical composition of any of embodiments 23-27, wherein the pharmaceutical composition is substantially free of another active ingredient for treating gout and/or lead nephropathy.

Embodiment 29—The pharmaceutical composition of any of embodiments 23-28, wherein the pharmaceutical composition is substantially free of sophoridine.

Embodiment 30—The pharmaceutical composition of any of embodiments 23-29, wherein the pharmaceutical composition is in the form of tablets, pills, capsules, powders, granules, suppositories, dragees, ointments, sprays, inhalants, emulsions, solutions, suspensions, syrups, and elixirs.

Embodiment 31—The pharmaceutical composition of any of embodiments 23-30, wherein the pharmaceutical composition is configured for administration by a route selected from the group consisting of oral, intravenous, intramuscular, intra-arterial, intramedullary, intrathecal, intraventricular, transdermal, subcutaneous, intraperitoneal, intranasal, enteral, topical, sublingual, and rectal administrations.

As used herein, the singular terms “a,” “an,” and “the” include plural referents unless the context clearly dictates otherwise. Thus, for example, reference to a compound can include multiple compounds unless the context clearly dictates otherwise.

As used herein, the terms “substantially,” “substantial,” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations, which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scopes of this invention.

What is claimed is:

1. A method for treating gout and/or lead nephropathy, comprising administrating to a subject in need thereof a pharmaceutical composition comprising a therapeutically effective amount of glutathione or a pharmaceutically acceptable salt thereof.

2. The method of claim 1, wherein the subject is a human subject.

3. The method of claim 2, wherein the subject is suffering from gout.

4. The method of claim 2, wherein the subject is suffering from lead nephropathy.

5. The method of claim 1, wherein the method comprises administrating to the subject about 1-20 grams of glutathione per day.

6. The method of claim 1, wherein the pharmaceutical composition comprises at least about 3 wt. % of glutathione.

7. The method of claim 1, wherein the pharmaceutical composition further comprises at least one pharmaceutically acceptable carrier or excipient.

8. The method of claim 1, wherein the pharmaceutical composition's active ingredient consists essentially of glutathione.

9. The method of claim 1, wherein the pharmaceutical composition is substantially free of another active ingredient for treating gout and/or lead nephropathy.

10. The method of claim 1, wherein the pharmaceutical composition is substantially free of sophoridine.

11. The method of claim 1, wherein the pharmaceutical composition is in the form of tablets, pills, capsules, powders, granules, suppositories, dragees, ointments, sprays, inhalants, emulsions, solutions, suspensions, syrups, and elixirs.

12. The method of claim 1, wherein the pharmaceutical composition is administrated by a route selected from the group consisting of oral, intravenous, intramuscular, intra-arterial, intramedullary, intrathecal, intraventricular, transdermal, subcutaneous, intraperitoneal, intranasal, enteral, topical, sublingual, and rectal administrations.

13. The method of claim 1, wherein the pharmaceutical composition is orally administrated one to three times daily.

14. The method of claim 1, wherein the serum uric acid or urate level in the subject is reduced by at least about 10% after 84 days of treatment.

15. The method of claim 1, wherein the blood lead level in the subject is reduced by at least about 10% after 84 days of treatment.

16. The method of claim 1, wherein the serum creatinine level in the subject is reduced by at least about 10% after 84 days of treatment.

17. The method of claim 1, wherein the volume of kidney stones or tophi in the subject is reduced by at least about 10% after 84 days of treatment.

18. The method of claim 1, wherein the volume of uric acid deposition in symptomatic joint or bursa of the subject is reduced by at least about 10% after 84 days of treatment.

19. A method for treating a disease or condition selected from the group consisting of chronic gouty arthritis, acute inflammatory arthritis, uric acid nephropathy, kidney stones, tophi, and hyperuricemia, comprising administrating to a subject in need thereof a pharmaceutical composition comprising a therapeutically effective amount of glutathione or a pharmaceutically acceptable salt thereof.

20. A pharmaceutical composition for treatment of gout and/or lead nephropathy, comprising a therapeutically effective amount of glutathione or a pharmaceutically acceptable salt thereof, wherein glutathione is the sole active ingredient of the pharmaceutical composition, and wherein the pharmaceutical composition is adapted for oral administration.
